(19) United States
(12) Patent Application Publication
(10) Pub. No.: US 2026/0256607 A1
(43) Pub. Date: Sep. 3, 2026

(54) HEART VALVE PROSTHESIS DELIVERY SYSTEM AND METHOD FOR DELIVERY OF HEART VALVE PROSTHESIS WITH INTRODUCER SHEATH AND LOADING SYSTEM

(71) Applicant: JenaValve Technology, Inc., Irvine, CA (US)

(72) Inventors: Stefan SCHRECK, Duvall, WA (US); Hussain RANGWALA, Villa Park, CA (US); Payam SAFFARI, Aliso Viejo, CA (US); Makensley LORDEUS, Phoenix, AZ (US); Vincent Ming Hai CHOW, Irvine, CA (US); Kevin Han Tang CHU, Tustin, CA (US)

(73) Assignee: JenaValve Technology, Inc., Irvine, CA (US)

(21) Appl. No.: 19/656,608

(22) Filed: Apr. 23, 2026

Related U.S. Application Data
(63) Continuation of application No. 17/377,254, filed on Jul. 15, 2021, now Pat. No. 12,616,593.
Application No. 17/377,254 is a division of application No. 16/099,793, filed on May 10, 2017, now Pat. No. 11,065,138.
(60) Provisional application No. 62/336,153, filed on May 13, 2016.
(60) Provisional application No. 62/491,391, filed on Apr. 28, 2017.

(51) Int. Cl. A61F 2/966; A61F 2/00; A61F 2/24; A61F 2/95

(52) U.S. Cl. CPC A61F 2/966; A61F 2/0095; A61F 2/243; A61F 2/2436; A61F 2002/9505; A61F 2/9522; A61F 2002/9665; A61F 2250/0062; A61F 2250/0064; A61F 2250/0092

(57) ABSTRACT

The present disclosure relates to a delivery catheter and the stepwise release of a stent from the catheter into the vasculature of a patient, as well as a loading device for a transcatheter heart valve (THV) prosthesis.

This application is related to U.S. Provisional Application No. 62/136,092 filed on Mar. 20, 2015, and U.S. Provisional Application No. 62/336,153 filed on May 13, 2016, the entire disclosures of which are incorporated herein by reference.

The present disclosure relates to a delivery system, a catheter system and a method for the minimally invasive application of prostheses to individuals in need thereof and a method and device for loading a prosthesis onto a catheter system and/or a delivery system.

### BACKGROUND

The present disclosure relates to the field of medical devices, in particular to a prosthesis which is transplanted into an individual In need thereof in order to re-establish proper body functions by way of minimally invasive methods and means applicable therefor.

Examples of prostheses that are placed by way of minimally invasive methods are stents and heart valves like aortic and mitral heart valves. Heart valves today are applied e.g. by the transapical, transfemoral, or subclavial route.

Usually the prosthesis is delivered to the implantation site by way of a delivery system also denoted a catheter. The requirements for the catheter by way of transfemoral delivery is more complex as compared with the transapical route because for the narrower, longer and more tortuous pathway.

One example of such a delivery system is disclosed in EP238797781. This patent describes a transfemoral catheter for the delivery of an aortic heart valve. The patent does not disclose nor suggest the features described herein.

Usually the prosthesis is radially compressed onto the catheter and crimped to a small size in order pass through the vasculature of the patient and to be delivered to the implantation site. The different systems known in the prior art use catheter systems with a profile of 18 to 26 French. Because of the potential detrimental effect of long-term crimping on the properties of the tissue leaflets, the prosthesis is crimped or loaded onto the catheter in the operating room just prior to use. Typically, the loading procedure is performed in the sterile field by a trained operator using a dedicated loading tool. This adds to the complexity of the implant procedure

Another consideration is the accurate positioning of the prosthesis into the final implant location. The beating heart causes the native valve annulus to move with the cardiac cycle. This creates a non-stationary target. In some cases, the natural heart beat is interrupted during placement of the prosthesis to create a stationary target. In some cases, the prosthesis is deployed in a stepwise fashion to better control the positioning of the prosthesis. In some cases, the catheter is designed to retract the prosthesis after partial release in case re-positioning of the prosthesis is desired.

Another consideration In the delivery is the maneuvering of the prosthesis by way of the catheter through the vasculature and its bends. The fact that the vasculature is typically narrow, and particularly at the aortic entry into the heart a substantive curve with a narrow angle has to be passed through, represents a substantial challenge for such a delivery procedure and device.

Yet another consideration is the diameter size of catheter system. The diameter size of the crimped prosthesis in the catheter for delivery through the vasculature of the patient may affect the implantation procedure and/or functioning of the prosthesis upon implantation. Many known systems do not achieve an adequate crimping size, and often the prosthesis tissue is negatively affected in known systems during the crimping procedure.

An object of the present disclosure is to provide for a simple and precisely operable delivery system for stents or prostheses, in particular heart valve prostheses which improve or avoid the disadvantages of prior art delivery systems.

On the basis of the problems referenced and outlined above, certain embodiments of the present disclosure may address the issue of delivering and positioning a specialized endoprosthesis for treating a narrowed cardiac valve or a cardiac valve insufficiency which realizes optimum positioning accuracy and anchoring of the emplaced device. In addition, the treatment of the narrowed cardiac valve or cardiac valve insufficiency may be achieved by way of a relatively simple procedure to enable routine treatment of narrowed cardiac valve or cardiac valve Insufficiency without major stress to the patient.

### EXEMPLARY OBJECTS OF THE DISCLOSURE

One exemplary object of some aspects of the present disclosure includes providing a catheter system for delivery of a prosthesis, e.g. a heart valve. In particular, wherein the heart valve can securely be loaded and crimped by the operator with minimal effort and skill.

Another exemplary object of some aspects of the present disclosure includes providing a catheter and delivery system for a prosthesis designed in a manner in order to facilitate the delivery of the prosthesis to the target site. In some: systems of the present disclosure, for example, maneuvering through the vasculature of a patient is possible with reduced or even without the disadvantages known in the prior art.

Another exemplary object of the present disclosure includes a step-wise liberation of the prosthesis in order to place the prosthesis correctly (e.g., with the proper position and/or orientation) at the target site, enable repositioning in this manner and/or fine tuning of the positioning procedure.

### SUMMARY

The present disclosure relates in at least one aspect to a catheter having a mechanism for the sequential release of a stent into the vasculature. The stent may be a self-expanding stent. The stent may be covered by a graft. The stent may contain a heart valve prosthesis. The vasculature may include a blood vessel. The vasculature may include a native heart valve. The vasculature may include the annulus of a native heart valve.

In some aspects, the present disclosure relates to a method of sequentially releasing a stent into the vasculature. The method may include in a first step releasing a first end of the stent from the catheter, the first end of the stent contacting the vasculature upon release. The method may include in a second step releasing the second end of the stent, which releases the entire stent from the catheter,

In some aspects, the present disclosure relates to a loading device and a method for loading a prosthesis, preferably a replacement heart valve prosthesis, on a catheter system.

In some aspects, the present disclosure relates to a method of loading a device onto a catheter and/or a delivery device.

### BRIEF DESCRIPTION OF THE DRAWINGS

Exemplary embodiments will be described with reference to the appended drawings below.

Of these are:

FIG. 1: a section side elevation of a first exemplary embodiment of the catheter,

FIG. 2: a side elevation of stent containing a heart valve.

FIG. 3: a sectioned side elevation of the first embodiment of the catheter retaining the stent.

FIG. 4: a sectioned side elevation of the first embodiment of the catheter during deployment step 1.

FIG. S. a sectioned side elevation of the first embodiment of the catheter after completing deployment step I.

FIG. 6: a sectioned side elevation of the first embodiment of the catheter during deployment step 2.

FIG. 7: a sectioned side elevation of the first embodiment of the catheter after completing deployment step 2.

FIG. 8: a section side elevation of a second exemplary embodiment of the catheter,

FIG. 9: a section side elevation of a second embodiment of the catheter after completion of deployment step 1,

FIG. 10: a section side elevation of a second embodiment of the catheter after completion of deployment step 2.

FIG. 11: a section side elevation of a third exemplary embodiment of the catheter containing a stent.

FIG. 12: a section side elevation of a third embodiment of the catheter after completion of deployment step 1.

FIG. 13: a valve prosthesis partially collapsed in an exemplary delivery system.

FIG. 14: a valve prosthesis fully collapsed in the delivery system.

FIG. 15: an embodiment of a storage container for the valve prosthesis and distal section of the delivery system.

FIG. 16: an alternative embodiment of a storage container for the valve prosthesis and distal section of the delivery system.

FIG. 17: a first embodiment of an attachment mechanism between the distal and proximal section of the delivery system.

FIG. 18: a second embodiment of the distal section of the delivery system and attachment mechanism to the proximal end of the delivery system.

FIG. 19; a third embodiment of the distal section of the delivery system and attachment mechanism to the proximal end of the delivery system prior to assembly.

FIG. 20: a third embodiment of the distal section of the delivery system and attachment mechanism to the proximal end of the delivery system partially assembled.

FIGS. 21-23: a pull-wire one-actuator release mechanism of a transcatheter heart valve (THV) and sequence of release.

FIGS. 24-26: a push-wire one-actuator release mechanism of a THV and sequence of release.

FIGS. 27-30: ferrule locking clips as safety feature useful against unintentional release of the THV from the catheter.

FIGS. 31-38: loading tool components and sequence of use for a THV prosthesis.

### DETAILED DESCRIPTION

In at least one aspect, the present disclosure relates to a catheter system for sequential deployment of a stent comprising a first retaining sleeve, a second retaining sleeve, a catheter shaft, and a stent holder, wherein the first sleeve is axially movable with respect to the catheter shaft, the second sleeve is axially fixed to the catheter shaft, the stent holder is axially movable with respect to the catheter shaft.

The various embodiments of the disclosure may address in an advantageous and unexpected manner one or more of the objects discussed above. In particular, in some aspects, the delivery, positioning and/or repositioning of a heart valve prosthesis may be facilitated.

In at least one aspect, the present disclosure relates to a method for sequential deployment of a stent wherein a 1st sleeve (11) of the catheter is moved followed by the movement of a stent holder (15) resulting in the liberation and directed deployment of the stent.

The “stent” as understood by the disclosure can comprise a valve, e.g. a heart valve which can be made of any known and useful tissue, e.g. it can be made of or comprise pericardial tissue. Such a combination can be denoted “prosthesis” or “heart valve prosthesis” in the sense of the disclosure.

The operation of one sleeve or sheath at the distal end by way of an actuator for deployment and positioning the prosthesis may be advantageous and/or may simplify the deployment procedure. Some aspects may have the advantage that also all movements of the movable parts of the catheter part wherein the prosthesis is mounted are effected in basically one direction. This also may apply for the stent holder (15) in connection with the 2nd end (101) of the prosthesis. Accordingly, the procedure may be simplified for the operator of the catheter, and the deployment of the prosthesis may present a lower risk of a wrong or sub-optimal deployment and positioning at the target site.

In some aspects, it may be particularly advantageous that the prosthesis release steps are effected by movement in one direction which may result in better controllability and a more precise positioning at the target site, e.g., because the catheter can be kept easier at the target site during the deployment procedure.

The design of some exemplary catheters herein, and the part(s) of the catheters whereon the prosthesis is mounted, may be particularly advantageous in transfemoral (TF) applications. It should be noted that the design of the catheter capsule carrying the prosthesis according to any examples herein may be combined with any handle design containing the engineering features to operate and actuate the particular sleeves and shafts described herein and used for deployment of the prosthesis. The handle may comprise or contain one or several actuating parts or features that actuate the capsule parts in a sequential manner in order to deploy and position the prosthesis precisely and to liberate the prosthesis in a controlled manner at the target site. A “capsule” in the sense of the present disclosure may comprise all components related to mounting, liberating, and deploying the prosthesis or the stent in a controlled manner.

The skilled person will appreciate that materials usually applied in catheter and delivery systems can also be used in any embodiments according to the present disclosure. For example, in a catheter system according to one or more embodiments of the present disclosure, an introducer sheath may be used, and the introducer sheath may be flexible and/or may comprise a flexible polymer, a hydrophilic coating, a PTFE liner, coil reinforcement and/or braid reinforcement.

In at least one embodiment of the catheter system according to the present disclosure the delivery means and steering means are releasably connectable,

Parts that are Introduced can be guided by a guide wire known in the art and made from materials as usually applied in the medical field. Usual ports may be applied for transfemoral use.

It can be advantageous for some aspects of the present disclosure if the tip of the catheter device is soft or semi firm (e.g., made of a soft or semi firm material) and/or for the tip of the catheter device to be bendable (e.g., made of a bendable material) in order to facilitate passage through the vasculature of the patient. Known materials can be used for such a flexible tip.

The catheter and its different sections may be made of appropriate materials as known in the art of catheter design. The materials may comprise, e.g., nitinol, steel, polymers, rubber, and/or Teflon®, and depending on the function of the catheter part the material may be accordingly chosen.

In one aspect the disclosure relates to a catheter system for sequential deployment of a stent or prosthesis comprising a first retaining sleeve, a second retaining sleeve, a catheter shaft, and a stent holder, wherein: the first sleeve is axially movable with respect to the catheter shaft, the second sleeve is axially fixated to the catheter shaft, the stent holder is axially movable with respect to the catheter shaft.

In another aspect the disclosure relates to a method for sequential deployment of a stent or prosthesis wherein a 1st sleeve (11) of the catheter is moved followed by the movement of a stent holder (15) resulting in the liberation and directed deployment of the stent or prosthesis.

In yet another aspect the disclosure relates to a system for repairing a cardiac valve comprising of a valve prosthesis, a distal segment of the delivery system, and a proximal segment of the delivery system,
the valve prosthesis being at least partially retained by the distal segment of the delivery system, the valve prosthesis and the distal segment of the delivery system being stored together in a liquid for transport, the proximal segment of the delivery system being stored dry for transport, means of connecting the distal segment of the delivery system to the proximal segment of the delivery system,

In yet another aspect the disclosure relates to a system for repairing a cardiac valve comprising of one or more configurations of a valve prosthesis, one or more configurations of a distal segment of the delivery system, and one configuration of the proximal segment of the delivery system, the one configuration of the proximal segment of the delivery system being configured such that it connects to the one or more configurations of the distal segment of the delivery system and can deploy one or more configurations of the valve prosthesis.

In yet another aspect the disclosure relates to a method for preparing a system for repairing a cardiac valve for use, the system comprising of a valve prosthesis, a distal segment of the delivery system, and a proximal segment of the delivery system, the method comprising of a first step of engaging the valve prosthesis with the distal segment of the delivery system and a second step of connecting the distal segment of the delivery system to the proximal segment of the delivery system.

In yet another aspect the disclosure relates to a method for preparing a system for repairing a cardiac valve for use,
the system comprising of a valve prosthesis, a distal segment of the delivery system, and a proximal segment of the delivery system, the method comprising of
a first step of engaging the valve prosthesis with the distal segment of the delivery system,
a second step of sterilizing the distal end of the delivery system together with the valve prosthesis,
a third step of sterilizing the proximal end of the delivery system separate from the distal end of the delivery system and the valve prosthesis,
a fourth step of connecting the distal segment of the delivery system to the proximal segment of the delivery system.

In one aspect of the present disclosure, the placement of a valve prosthesis Is considered that consists of a radially collapsible and expandable stent segment and axially oriented support struts. The support struts engage with the native cusps of the diseased aortic valve. Embodiments of such a valve prosthesis are disclosed in patent application WO2011/147849. When placed in the native valve, the expandable stent segment creates a first anchoring force in the radial direction. The support struts create a second anchoring force in the axial direction, Placing such a valve prosthesis at the implant location may include first engaging the support struts with the cusps of the native valve, secondly expanding the expandable stent segment in the native annulus, and finally releasing the remainder of the prosthesis from the delivery catheter. The figures describe exemplary embodiments of the disclosure without to be understood as limiting. Any aspect or feature as disclosed in each of the figures shall be understood as being combinable with all and any other aspects or features of all figures described and depicted in this disclosure.

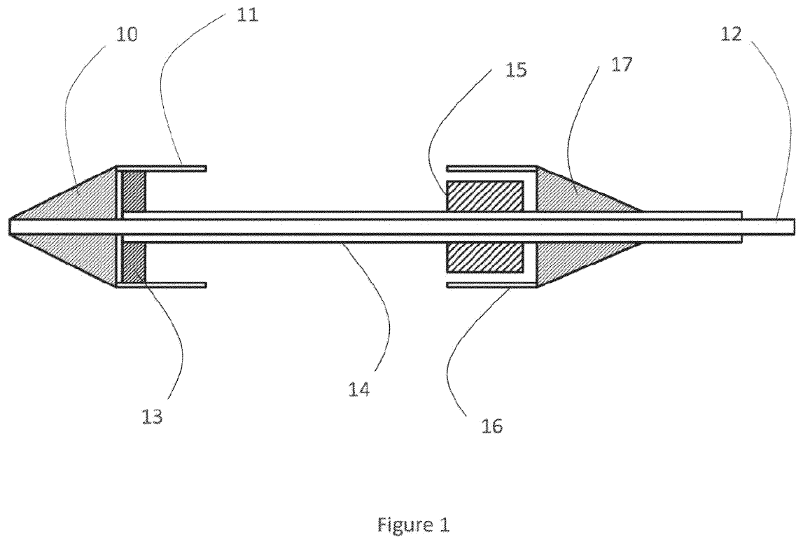

FIG. 1 shows a section side elevation view of the first exemplary embodiment of the catheter. The catheter comprises a nose cone (10) and a first sleeve (11) connected to the nose cone (10). The nose cone (10) is also connected to a first shaft (12). The first shaft (12) slides within a second catheter shaft (14), A front stop (13) is mounted at the first end of the second catheter shaft (14). A back cone (17) is mounted onto the second shaft (14) and a second sleeve (16) is connected to the back cone (17). A stent holder (15) sits on the second shaft (14). The stent holder (15) can slide freely along the second shaft (14). A back mount (17) is firmly connected to the second shaft (14), A second sleeve (16) is connected to the back mount (17).

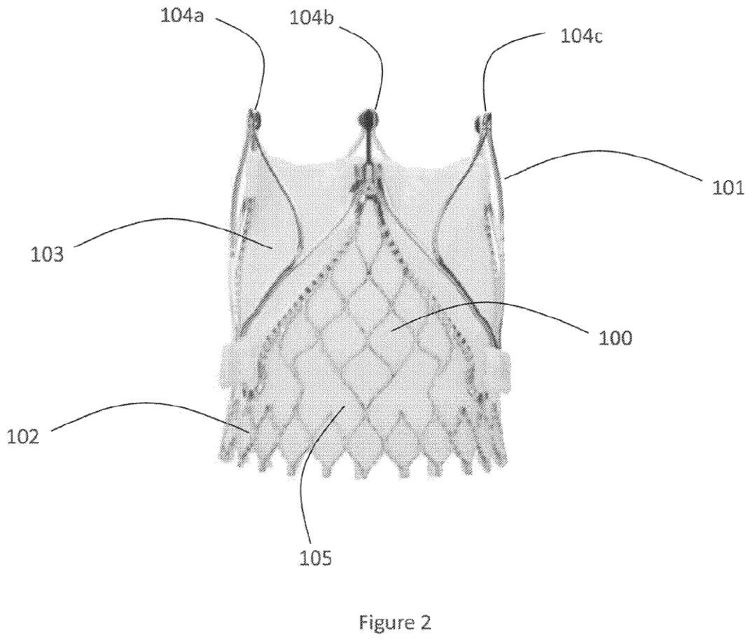

FIG. 2 shows an example of a stent that can be delivered by the catheter in FIG. 1. The stent (100) consists of a first end (102) and a second end (101), The second end (101) may contain eyelets (104a-c) to secure the stent to the delivery system. The stent (100) contains a bioprosthetic valve (103). The first end (102) of the stent (100) is covered by pericardial tissue (105), It is understood that there are many possible embodiments of a stent, and the present disclosure includes other types of stents and stent designs than shown, The stent may be a bare stent, the stent may be a covered stent, the stent may contain a prosthetic valve for replacement of an aortic, mitral, tricuspid, pulmonic, or venous valve. The stent may be braided, The stent may be cut from a metal tube. The stent may be self-expanding. The stent may be actively expanded.

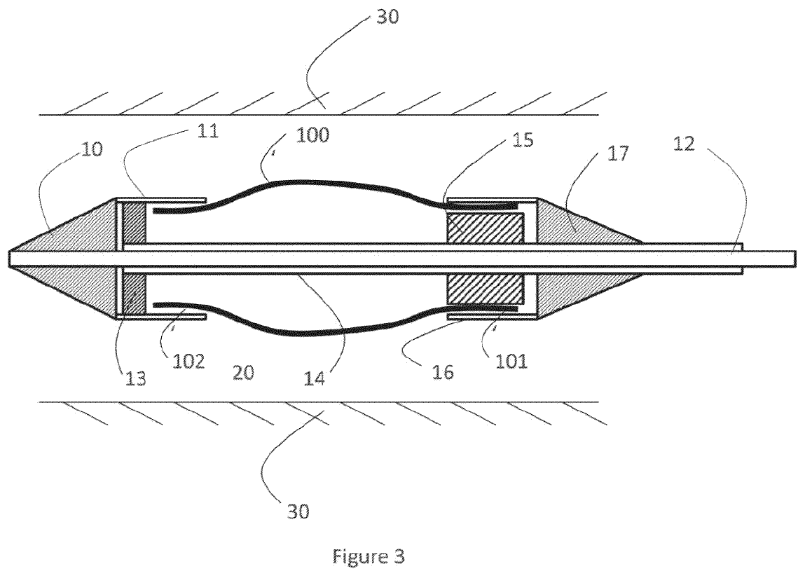

FIG. 3 shows the stent (100) mounted in the catheter of FIG. 1, The first end of the stent (102) is retained by the first sleeve (11). The second end of the stent (101) is retained by the second sleeve (16). A stent holder (15) supports the second end of the stent (102), The stent (100) is sandwiched between the second sleeve (16) and the stent holder (15). The stent holder (15) may contain recesses to engage with the eyelets of the stent (105a-c in FIG. 2). The catheter is positioned in the target vessel (30). The first sleeve (11) and the second sleeve (16) may only partially cover the stent (100) as shown in FIG. 3. The first sleeve (11) and the second sleeve (16) may together completely cover the stent (100). The first sleeve (11) may overlap with the second sleeve (16).

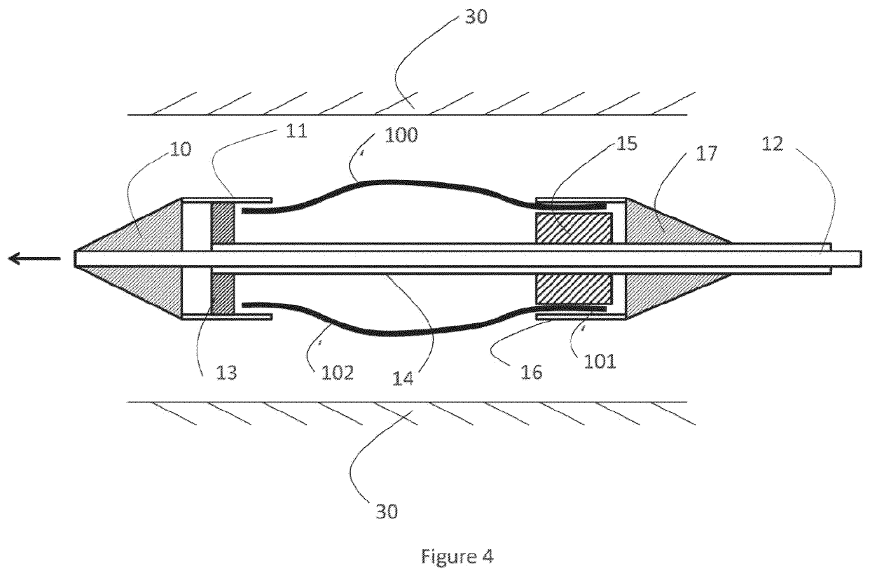

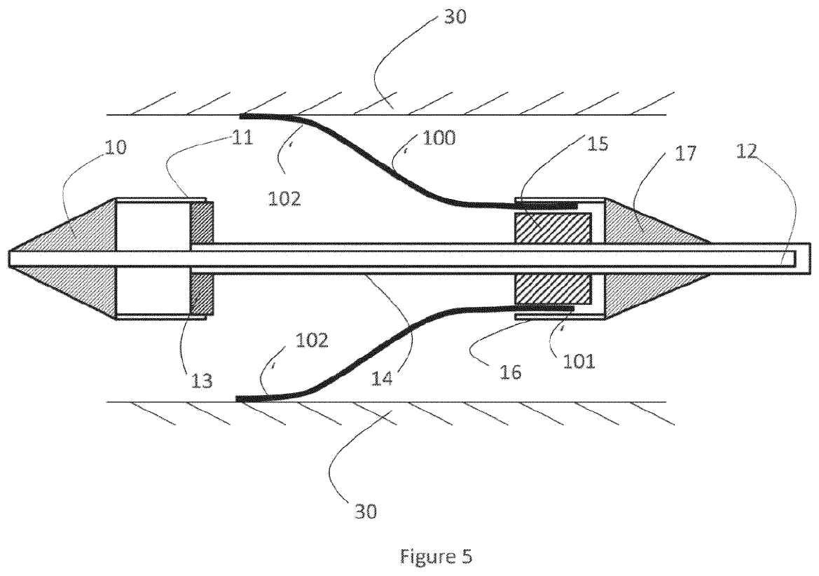

FIG. 4 shows the delivery catheter of FIG. 1 during the first step of the deployment. The first shaft (12) is advanced distally (toward the tip of the catheter), moving the nose cone (10) and first sleeve (11) distally, The front stop (13) prevents the stent (100) from moving distally, As a result, the first sleeve (11) releases the first end of stent (100).

FIG. 5 shows the delivery catheter of FIG. 1 at the end of the first step of the deployment. The first sleeve (11) is advanced distally until the first end of the stent (102) is fully released from the first sleeve (11), The unrestrained first end of the stent (102) expands and contacts the walls of the target vessel (30). The second end of the stent (101) is retained by the second sleeve (16) and the stent holder.

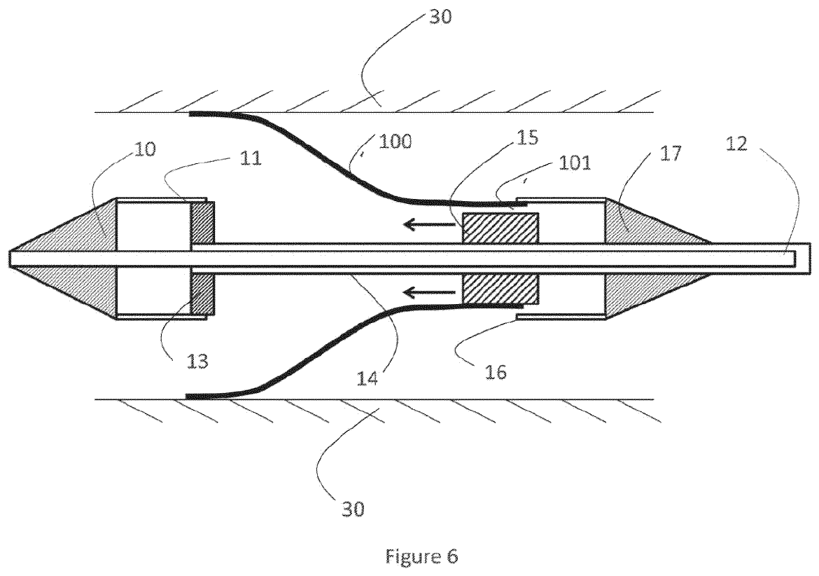

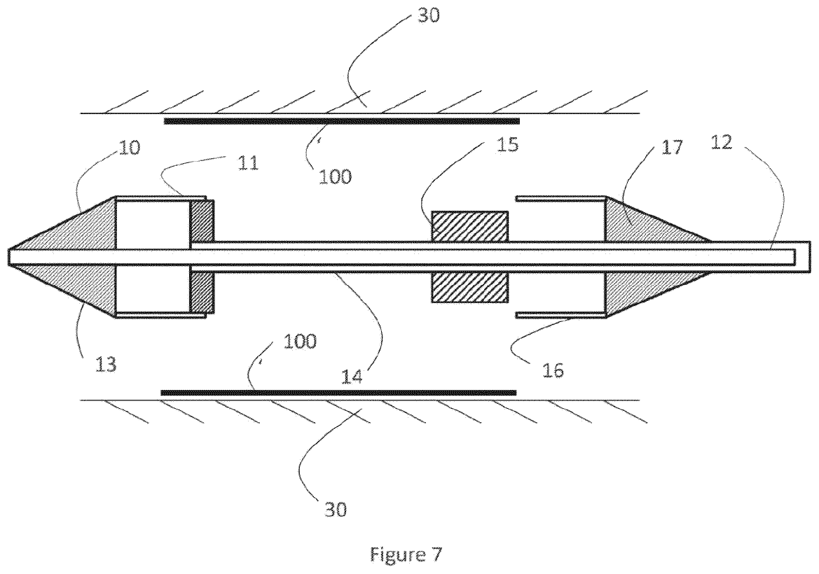

FIG. 6 shows the initiation of the second step of deployment. The stent holder (15) can slide freely along the second shaft (14). Once the first end of the stent (102) is released, the second end of the stent (101) in conjunction with the stent holder (15) can move distally with respect to the second sleeve (16) and the second shaft (14). The movement of the stent (100) and stent holder (16) may be initiated by pulling the second shaft (14) proximally. The first end of the stent (102) is fixed against the walls of the target vessel (30) preventing the stent (100) and the stent holder (16) from moving with the second shaft (14), The distal movement of the stent (100) and stent holder (16) with respect to second shaft (14) causes the second end of the stent (101) to release from the second retaining sleeve (16), FIG. 7 shows the stent (100) fully released from the catheter.

In certain circumstances, active movement of the second shaft (14) proximally may not be required to release the stent (100), The expansion forces of the stent (100) may be sufficient to pull the second end of the stent (101) and the stent holder (15) from the second sleeve (16). Furthermore, the wall of the target vessel (3) may move with the cardiac cycle. For example, the aortic annulus typically moves 0.5 mm to 2 mm axially with every heartbeat. The first end of the stent (102) once anchored to the annulus after completion of the first deployment steps moves with the annulus. This movement may by itself or in conjunction of the expansion forces of the stent (100) may be sufficient to pull the second end of the stent (101) and the stent holder (15) from the second sleeve (16).

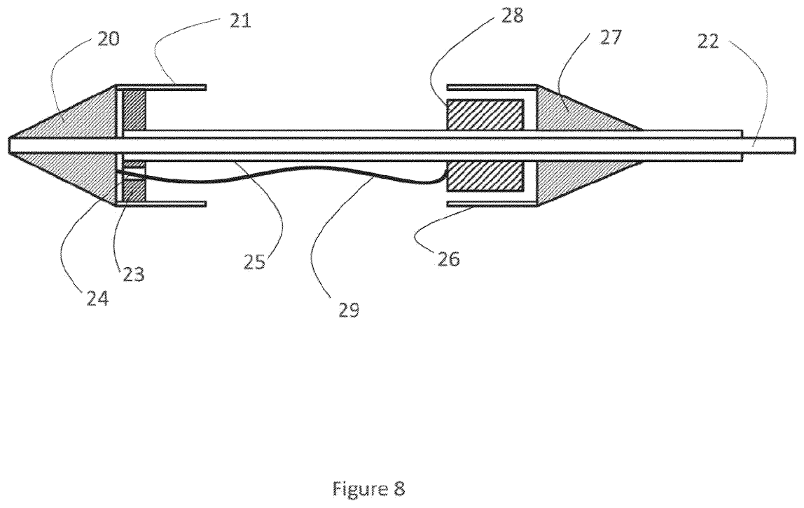

FIG. 8 shows another exemplary embodiment of the delivery catheter. The catheter comprises a nose cone (20) and a first sleeve (21) connected to the nose cone (10). The nose cone (20) is also connected to a first shaft (22), The first shaft (22) slides within a second catheter shaft (25). A front stop (23) is mounted at the first end of the second catheter shaft (25), The front stop contains a passage (24). A back cone (27) is mounted onto the second shaft (25) and a second sleeve (26) is connected to the back cone (27). A stent holder (28) sits on the second shaft (25). The stent holder (28) can slide freely along the second shaft (25). A cable (29) connects the stent holder (28) to the nose cone (20). The cable (29) passes through the passage (24) in the front stop (23), The cable (29) is longer than the distance between the stent holder (28) and the nose cone (20). A back mount or back cone (27) is firmly connected to the second shaft (25). A second sleeve (26) is connected to the back mount (27).

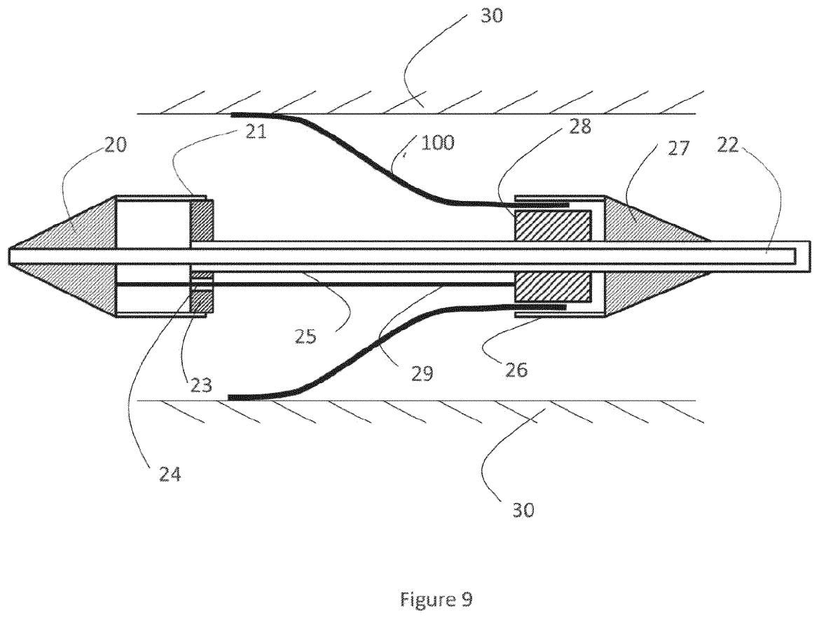

FIG. 9 shows the delivery catheter of FIG. 8 after completion of the first deployment step. The first shaft (22), nose cone (20), and the first sleeve (21) are advanced distally, which released the first end of the stent (102). In this configuration, the distance between the stent holder (28) and the nose cone (20) is approximately equal to the length of the cable (29), The cable (29) is fully extended.

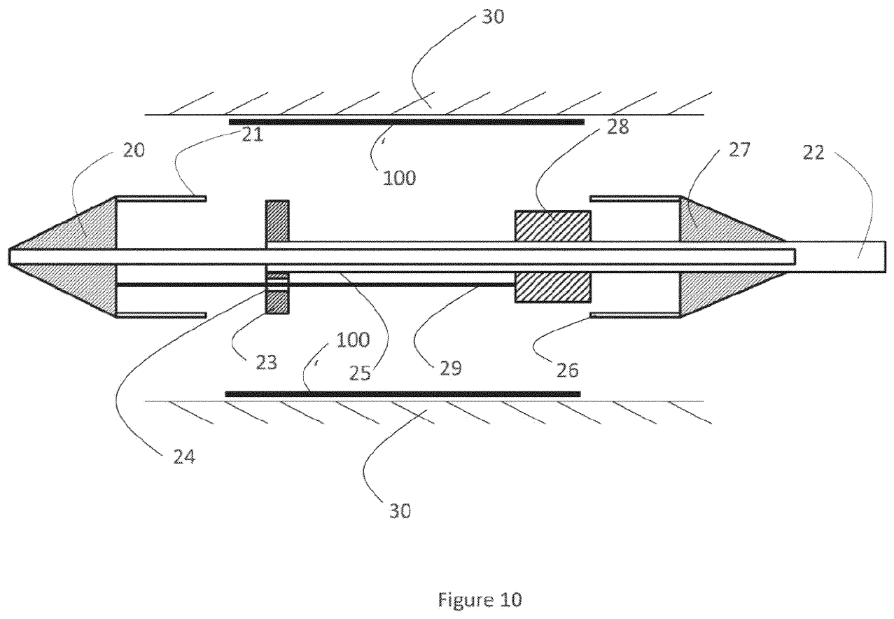

FIG. 10 shows the delivery catheter of FIG. 8 after completion of the second deployment step. The first shaft (25), nose cone (20), and the first sleeve (21) are further advanced distally. The cable (29) pulls the stent holder (28) distally and out of the second sleeve (26). The second end of the stent (101) is released from the catheter. The cable (29) provides the means for a sequential release of the first end (102) and the second end (101) of the stent (100) by a single movement of the first shaft. Other means of connecting the front assembly consisting of the nose cone (20), the first sleeve (21), and the first shaft (22), with the stent holder (28) may be considered and used herein. The connecting element may be a tension spring that when placed under tension after completion of the first deployment step pulls the stent holder (28) distally. The connecting element may be a rode that is fixed to the nose cone (20) and can pass through a channel in the stent holder (28). The end of the rode that passes through the channel has an enlarged end cap that cannot pass through the channel. As the rode is pulled distally by the front assembly, the end cap catches the stent holder (28) at the end of the first deployment step and pulls the stent holder (2) distally during the second deployment step,

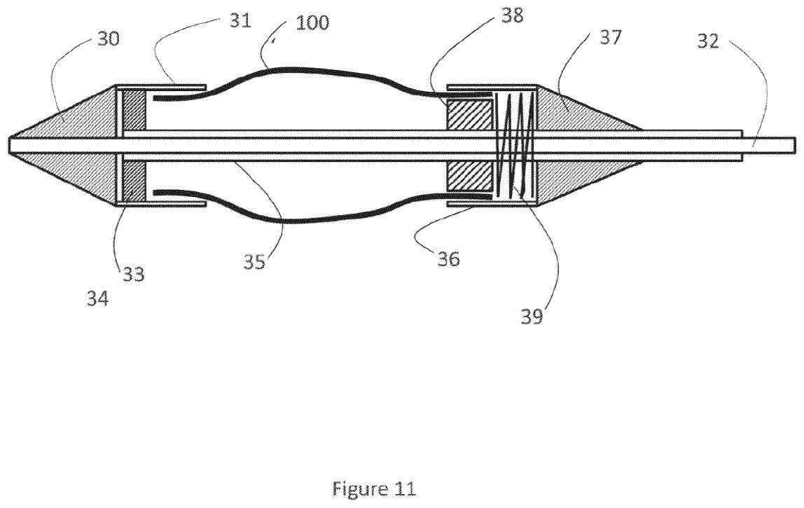

FIG. 11 shows yet another exemplary embodiment of the delivery catheter. The catheter comprises a nose cone (30) and a first sleeve (31) connected to the nose cone (30). The nose cone (30) Is also connected to a first shaft (32), The first shaft (32) slides within a second catheter shaft (35), A front stop (33) is mounted at the first end of the second catheter shaft (35). A stent holder (38) sits on the second shaft (35). The stent holder (38) can slide freely along the second shaft (35). A back mount (37) is firmly connected to the second shaft (35). A second sleeve (36) is connected to the back mount (37), A compression spring (39) is mounted on the second shaft (35) between the stent older (38) and the back mount (37). The stent (100) is retained by the first sleeve (31) and the second sleeve (36).

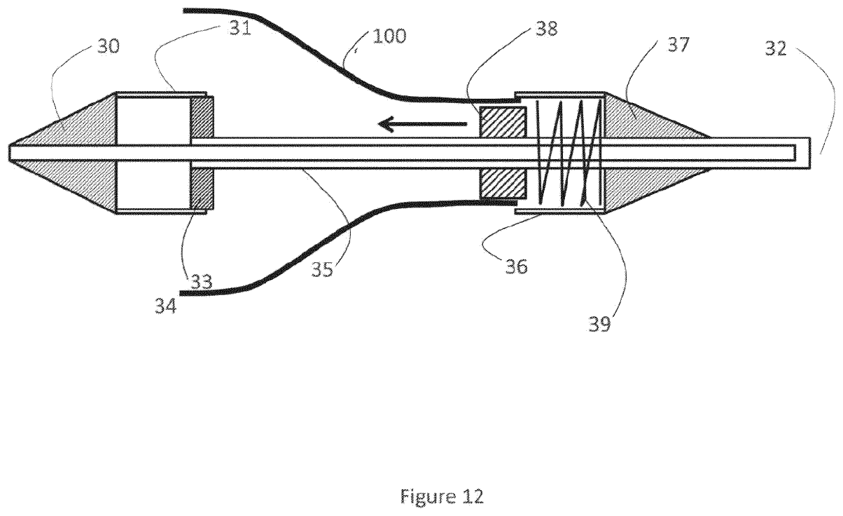

FIG. 12 shows the catheter of FIG. 11 after completion of the first deployment step and initiation of the second deployment step. The first shaft (32), nose cone (30), and the first sleeve (31) are advanced distally, which releases the first end of the stent (102). In this configuration, the spring (39) pushes the stent holder (38) from the second sleeve (36) which then releases the second end of the stent (101) from the catheter.

The catheter or delivery system as disclosed herein can be characterized by the above features alone or in combination with the below features, or it can be characterized by any of the below features alone or any combination thereof or a combination of the above features and any of the below described features.

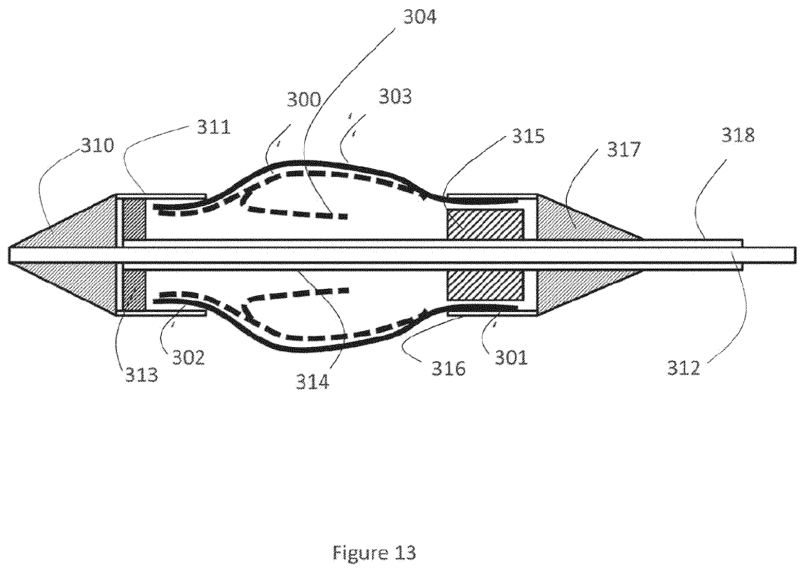

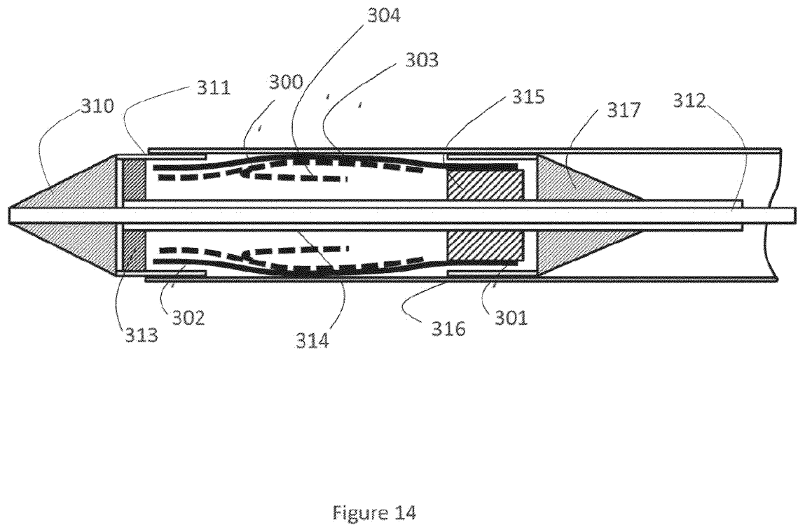

Typically, transcatheter valve prostheses are stored separately from the delivery system in a liquid solution to preserve the animal tissue, which form the leaflets of the valve prosthesis. The valve prosthesis is loaded onto the delivery system in the operating room immediately before use. Special loading tools and precise instructions and training of the operator are necessary to ensure proper loading. In another aspect of the present disclosure, pre-loading of the valve prosthesis onto the delivery system before packaging and shipment to hospital is considered. FIG. 13 shows a cross-sectional view of the valve prosthesis crimped in the delivery system described in FIGS. 3-7. The distal end of the delivery system comprises or consists of a nose cone (310), a first sleeve (311) connected to the nose cone (310), a first shaft (312) connected to the nose cone (312), a second sleeve (316) connected to a back cone (317), a second shaft (318) coaxial and external to the first shaft (312) connected to the back cone, a front stop (313) connected to the second shaft (318), and stent holder (315), The stent holder (315) can move axially with respect to the second shaft (318). The first shaft can move axially within the second shaft (318). The proximal end (302) of the valve prosthesis (300) is retained by the first sleeve (311) and the distal end (301) of the valve prosthesis (300) is retained by the second sleeve. The midsection (303) of the valve prosthesis (300) is unconstrained and has a diameter larger than the crimped distal end (301) and proximal end (302). The tissue leaflets (304) which are located in the midsection (303) of the valve prosthesis (300) are slightly folded but not crimped. In FIG. 14, the valve prosthesis (300) is fully collapsed for insertion into the patient. The midsection (303) of the valve prosthesis is constrained by the introducer sheath (316) and the tissue leaflets (304) are fully crimped.

The current disclosure contemplates shipping and storing the valve prosthesis in the configuration shown in FIG. 13. In the operating room prior to use, the operator slides the introducer sheath (316) over the midsection (303) of the valve prosthesis to prepare the system for insertion in the patient. It is further contemplated to separate the distal end of the delivery system, which houses the valve prosthesis, from the remaining proximal section of the delivery system, The valve prosthesis is pre-loaded onto the distal end of the delivery system and stored in one container, The proximal segment of the delivery system is stored in a second container. Prior to use the two components of the delivery system are connected.

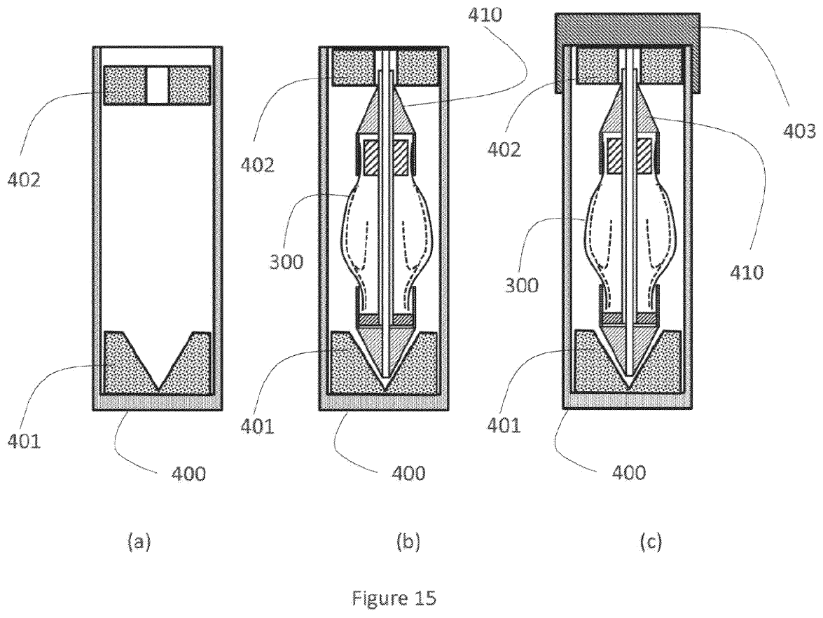

FIG. 15 shows one embodiment of the storage container (400) for the pre-loaded valve prosthesis (300). The container (400) is designed to contain a liquid storage solution to maintain sterility and prevent dehydration of tissue leaflets. The storage solution may contain gluteraldehyde, formaldehyde, alcohol, TWEEN, and other chemical(s) suitable for storing cross-linked tissue. The tissue may be harvested from an animal. The tissue may be harvested from the animal's pericardium, blood vessels, cardiac valve, or Intestines. The container (400) contains a distal holder (401) and a proximal holder (402). The distal end of the delivery system (410) retains the proximal section (302) and distal section (301) of the valve prosthesis (300). A lid hermetically (403) seals the container (400).

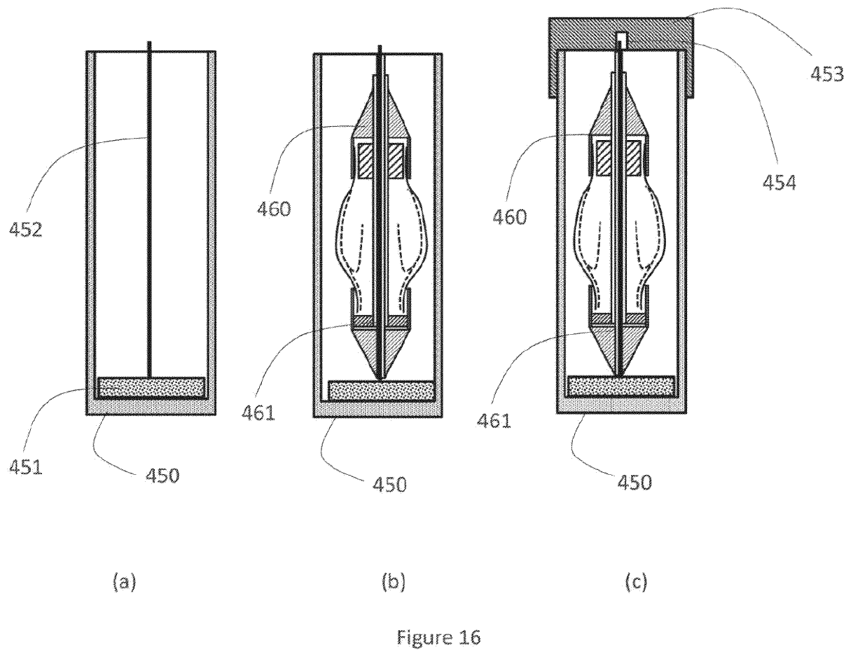

FIG. 16 shows an alternative embodiment of the storage container. The container (450) contains a distal holder (451). A rode (452) is connected to the distal holder (451). The rod (452) passes through the guidewire lumen (461) of the distal segment of the delivery system (460) and centers the distal segment of the delivery system (460) in the container (450). A lid (453) hermetically seals the container (450). A central recess (454) in the lid (453) stabilizes and centers the rod (452).

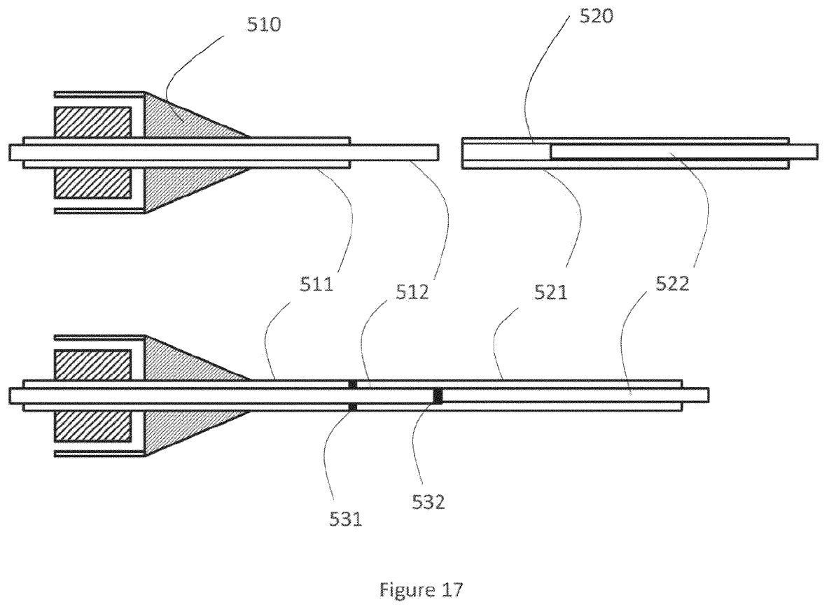

FIG. 17 Illustrates exemplary means of connecting the distal segment of the delivery system (510) to the proximal segment of the delivery system (520), To make the assembled delivery system fully operable, the outer shaft (521) of the proximal segment (520) is connected to the outer shaft (511) of the distal segment (510) of delivery system by an outer connector (531) and the inner shaft (522) of the proximal segment (520) is connected to the inner shaft (512) of the distal segment (510) of delivery system by an inner connector (532). The means of connection may include, but are not limited to, a male-female luer connector, a male-female thread, an interference press fit between the proximal and distal shaft, an adhesive, magnets, barbs, clips, combinations thereof, or any other suitable means of connecting two tubular segments.

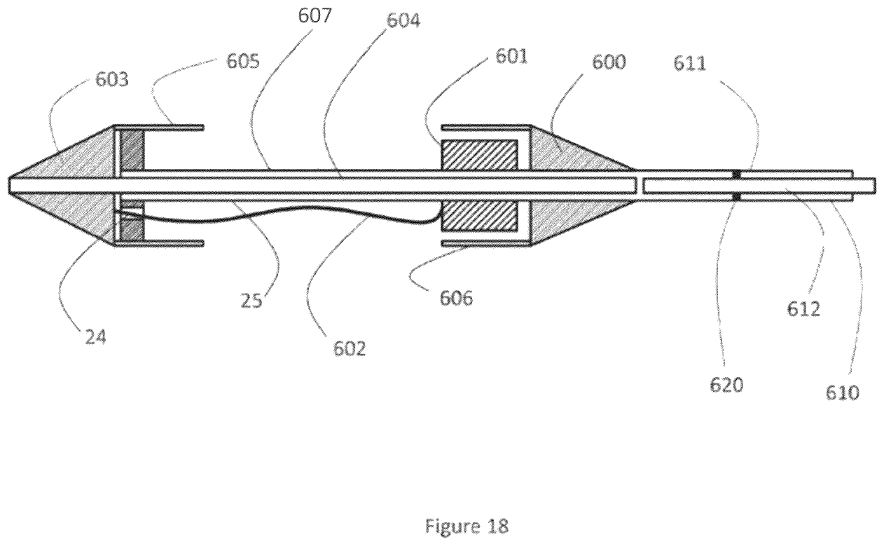

FIG. 18 shows an embodiment of the delivery system previously described in FIG. 8. In this embodiment, the stent holder (601) is connected with a cable, wire or string (602) to the nose cone (603) of the delivery system. The inner shaft (604) of the distal delivery system segment (600) is connected to the nose cone (603). Advancing the Inner shaft (604) distally sequentially moves the first sleeve (605) distally to release the proximal end of the valve prosthesis and then the stent holder (601) to release the distal end of the valve prosthesis from the second sleeve (606). To operate the distal segment of the delivery system (600) from the proximal end (610) of the delivery system, the outer shaft (607) of the distal segment of the delivery system (600) is connected to the outer shaft (611) of the proximal segment of the delivery system (610) by a first connector (620). The inner shaft (604) of the distal segment of the delivery system (600) is axially aligned but not physically connected with the inner shaft (612) of the proximal segment of the delivery system (610). Advancing the inner shaft (612) of the proximal delivery system (610) also advances the inner shaft (604) of the distal segment of the delivery system (600). The advantage of the embodiment of FIG. 18 is that only a single connector is needed to assembly the proximal and distal segment of the delivery system,

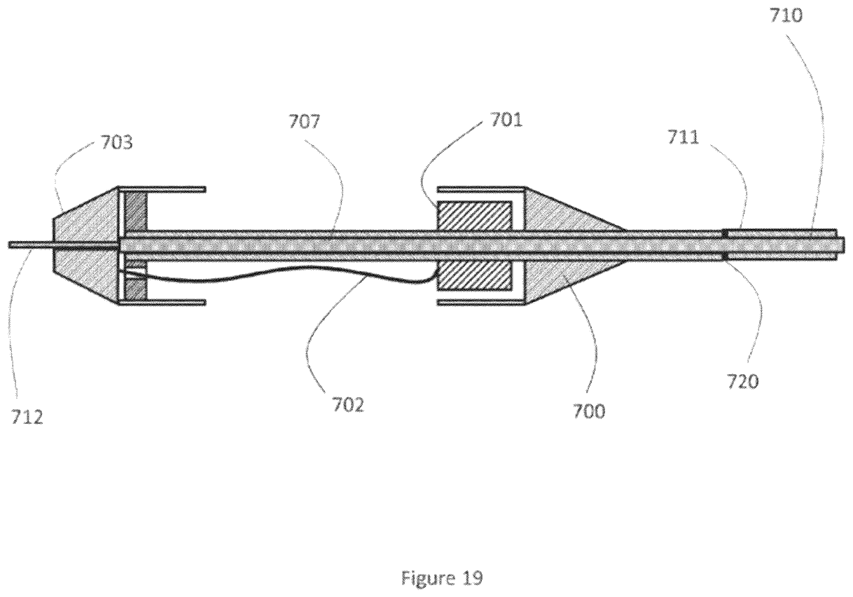

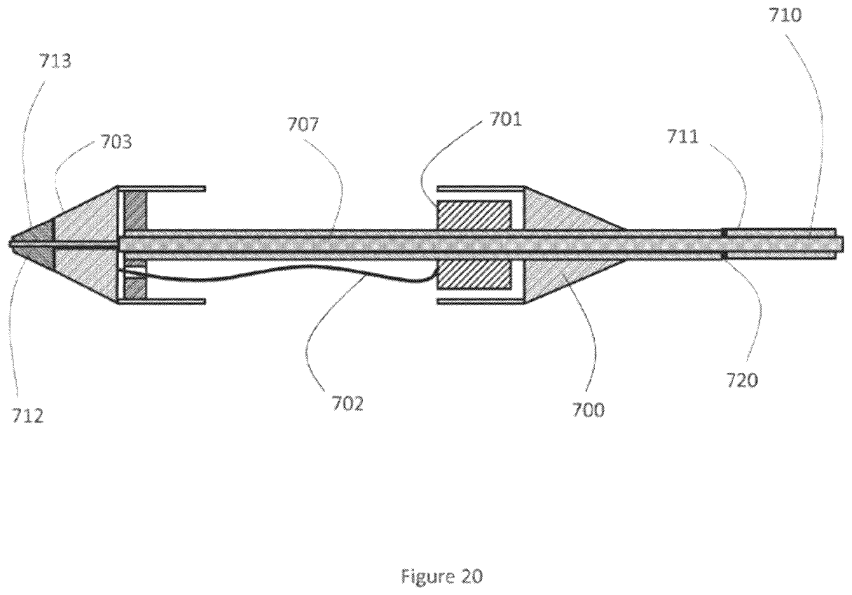

FIGS. 19 and 20 show another embodiment of the two-segment delivery system. In this embodiment, the stent holder (701) is connected with a wire or string (702) to the nose cone (703) of the delivery system as shown in FIG. 19. To operate the distal segment of the delivery system (700) from the proximal end (710) of the delivery system, the outer shaft (707) of the distal end (700) of the delivery system is connected to the outer shaft (711) of the proximal delivery system (710) by a first connecting means (720), The inner shaft (712) of the proximal segment (710) passes through the outer shaft (707) and the nose cone (703) of the distal segment of the delivery system (700). The outer diameter of the inner shaft (712) is reduced as it passes through the nose cone (703) to create a physical interference between the inner shaft (712) and the nose cone (703) at the proximal end of the nose cone (703). FIG. 20 shows the final assembly of the distal segment of the delivery system (700) and the proximal end of the delivery system (710), A nose tip (713) is connected to the distal end of the inner shaft (712) to secure the nose cone to the inner shaft (712), Advancing the Inner shaft (712) of the proximal segment of the delivery system (710) distally advances the nosed cone (703), the first sleeve (705) followed by the stent holder (701) similar to the embodiment in FIG. 18.

Valve prostheses are provided in several sizes to treat the wide range of valve anatomies found in patients. For example, aortic valve prostheses may be provided in diameters ranging from 19 mm to 29 mm. Different sizes of valve prosthesis often require different size delivery systems. For example, larger diameter valves may require larger diameter sleeves, Often the length of the valve prosthesis increases with diameter requiring longer pockets and longer sleeves to retain the valve prosthesis. In a further aspect of the current disclosure, a single configuration of the proximal segment of the delivery system is envisioned that can mate and connect to different configurations of the distal delivery system. Since the distal segment of the delivery system is stored with the valve prosthesis, a single proximal segment of the delivery system can be used for all valve sizes. This reduces the Inventory of delivery systems needed in the hospital.

Additional features which may serve Inter alia to simplify the deployment procedure of a prosthesis in e.g. a percutaneous deployment method may include one or several of the following: a spring loaded eyelet holder, a spring loaded rombi sleeve, a tension spring in the handle of the catheter, a sliding lock, a locator spread, a retraction protector for withdrawal of the catheter, a quick release steering which may include a push wire release and/or an integrated sheath, a cartridge holding the prosthesis, preferably in a preloaded manner. The catheter may be designed in a manner so that each of the above elements are coordinated functioning depending upon their inclusion in the device.

A deployment of a prosthesis may be characterized by one or more of the following steps depending on the inclusion of the above described design features,

Firstly, the prosthesis is loaded onto the capsule of the catheter as described below using a specifically designed loading device. In case of a preloaded prosthesis the unit carrying the prosthesis is combined with the catheter, e.g, by way of a click mechanism, Otherwise the prosthesis is loaded onto the catheter as e.g. described below,

The catheter including the prosthesis is introduced Into the vasculature of a patient and pushed upwardly in direction of the heart. After reaching the heart the catheter portion carrying the prosthesis is centered—in case of an aortic heart valve—to be proximal to and essentially in the center of the endogenous aortic heart valve. This centering can be achieved by way of a steering mechanism included in the catheter. In a first step the prosthesis is partially released and in case the prosthesis is exhibiting one or more feelers or locators these parts of the prosthesis are pushed into the pockets of the endogenous valve, This will support a correct positioning of the prosthesis in the correct position. Moreover, the commissures of the prosthesis can be aligned with the endogenous commissures in order to achieve a positioning which is similar to the endogenous symmetry of the valve. In a next step the tip lock (safety clip) is taken off the device and is pushed distally, The proximal part of the catheter holding the prosthesis is thereby moved also distally which can be achieved by a wire connecting the distal and proximal part (stop) carrying the prosthesis. In this manner the proximal part of the prosthesis held in place by a sheath will be released in a second step as a consequence of pushing the distal part (stop) distally. This sequence of deployment steps describes a transfemoral deployment procedure.

In a transapical application of the device, the parts of the catheter and the prosthesis are placed 180° in the other direction.

In an exemplary device as disclosed herein the prosthesis is held and released (deployed) wherein the device has a first movable sleeve and a stop (also denoted as crown or prosthesis holding means) and a second sleeve which is stationary and wherein the stop is movable.

The release of the prosthesis is achieved by way of a release spring and the release is triggered by a release not actuating the release spring. In known devices, the prosthesis is released by way of an actuation from the handle to the catheter part carrying the prosthesis by way of a catheter sleeve. This implied the application of a force of a long distance and wherein the catheter shaft is bent.

Accordingly, the transmission of the force applied is problematic. There may even occur cases where the force cannot be transmitted and as a result the prosthesis is not released and jams. The advantage of a design and release mechanism according to some aspects of the present disclosure is that the release procedure is not only more direct and such forces are no longer required but that the release procedure is more reliable.

The release procedure can also be denoted a “single release” mechanism. Advantage of such a single release is that it avoids friction in the system which may be problematic. It involves less movable parts and thus implies less risk of malfunction. The system as disclosed implies less steps and accordingly is more convenient for the operator to use it. The catheter shaft contains less parts, and thus the system may be more flexible and easier to operate through the vasculature. In view of the lower number of parts also less safety parts are required, which may simplify the procedure and/or increase the success rate. Thus operator mistakes are avoided.

The catheter can be combined with a sheath system which may be preshaped and can be designed to be steerable, The sheath may be have a hydrophilic coating.

The outer diameter may be 20 F to 22 F, preferably 21 F, and the inner diameter may have a size 18 F to 20 F, preferably 19 F. The outer sheath may exhibit a click mechanism to combine it with the catheter and to release it again after deployment of the prosthesis.

The catheter may comprise the following features at its distal part where the prosthesis is loaded. The catheter may comprise a flexible tip, a sealing ring sleeve to hold the distal region of the prosthesis, a retraction taper, a control wire, a proximal stent holder means, preferably with openings for holding the eyelets of the prosthesis, and a ferrule sleeve, which parts are connected with a handle for actuating and operating the deployment procedure.

The handle comprises a release mechanism which actuates the prosthesis deployment and exhibits a guidewire luer. The sealing ring release spring is secured by means of a tip lock which can be taken off the handle for deployment.

The catheter containing the loaded prosthesis can be introduced by means of a transition tube into the outer sheath and introduced into the vasculature up to the heart.

A prosthesis which is deployed by use of the above described catheter and delivery system can be loaded by generally known means or specifically adapted devices and methods known in the art. Moreover, an exemplary specifically designed loading device will be described in the following. The prosthesis as disclosed herein can also be stored and/or transported in a special device as described below containing a solution which serves inter alia to protect the prosthesis.

In another aspect the disclosure relates to a specific release mechanism useful for deployment of the prosthesis at its target site in the patient's heart.

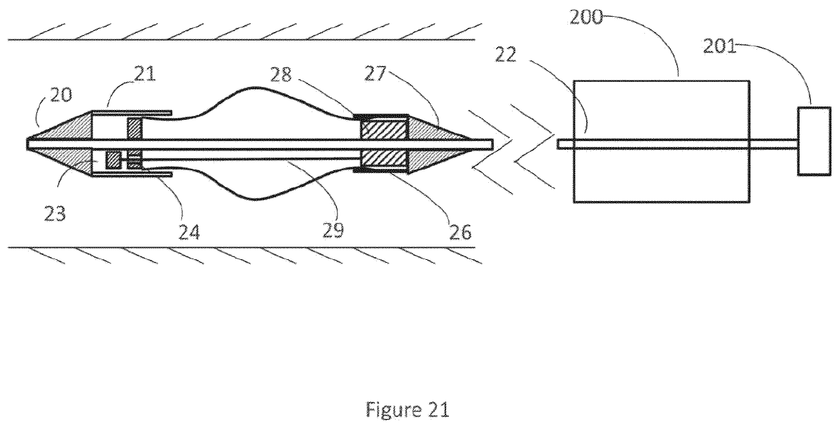

In FIG. 21, the bend stylized prosthesis is mounted on the distal part of the delivery system, First sleeve (21) covers the distal part of the prosthesis and a second sleeve (26) covers and holds the proximal part of the prosthesis wherein back mount (27) and nose cone (20) are located proximal and distal, respectively. The pull wire or cable (29/29a) connects the proximal stent holder (28) and the distal part as indicated by reference numbers (23) and (24). The handle (200) is connected by way of first shaft (22) with the nose cone (20) passing through stent holder (28). Plunger (201) serves to actuate the release of the prosthesis.

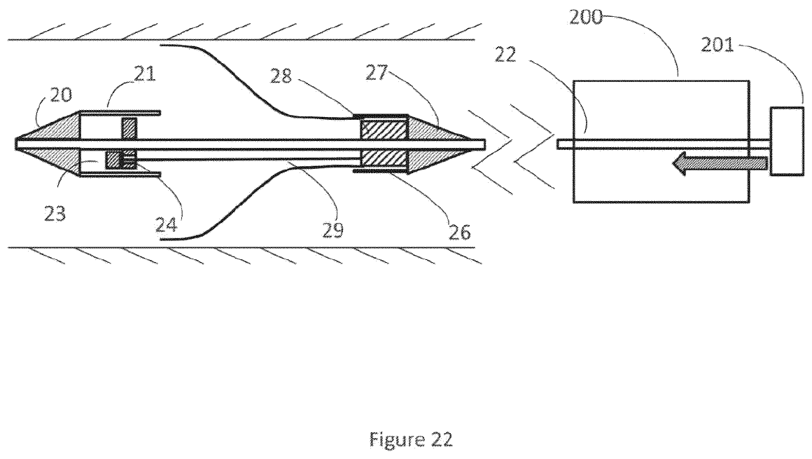

In FIG. 22 plunger (201) is pushed distally whereby the first sleeve (21) releases the distal part of the prosthesis. Front stop (23) moves closer to front stop and the passage indicated (24). The arrow Indicates the movement.

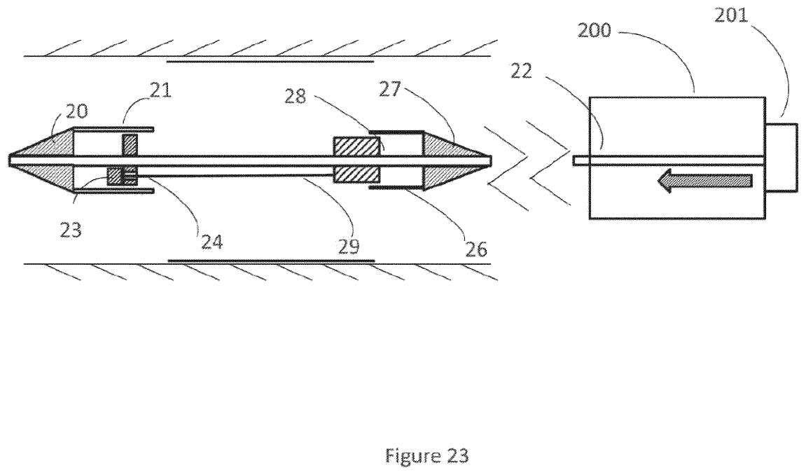

In FIG. 23 the plunger (201) is pushed further in distal direction and thus moves the nose cone (20) further distally whereby pulling by cable (29/29a) the stent holder (28) further in distal direction. The second sleeve(26) connected to the back mount (27) remains static whereby the proximal part of the prosthesis is released fully. The arrow indicates the movement. Thus by way of one actuator a two-step release and deployment sequence can be achieved wherein one sleeve (21) is moved and a second sleeve (26) is being kept static wherein the front part of the catheter is moved distally and leads to a complete release of the prosthesis.

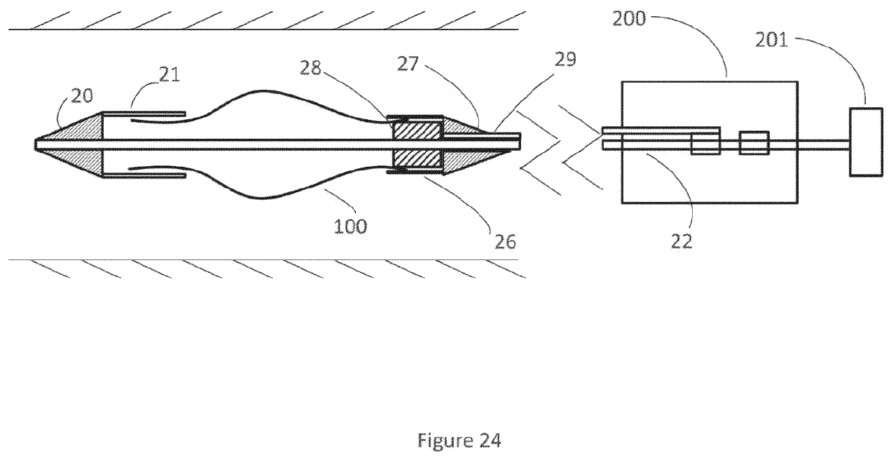

In FIGS. 24-26, the release and deployment of the prosthesis is performed in the same sequence as in FIGS. 21-23, however, by way of a push mechanism. In FIG. 24, the handle (200) comprises a plunger (201) which moves a first shaft (22). A shaft (29/29b) is connected with proximal stent holder (28). The prosthesis is mounted in the capsule by way of a distal first sleeve (21) and a proximal second sleeve (26). The stent holder (28) is connected to the handle to a stop. The plunger (201) can actuate a first shaft (22) to which a stop is connected in the handle and which first shaft (22) is also connected with the nose cone and first sleeve (21).

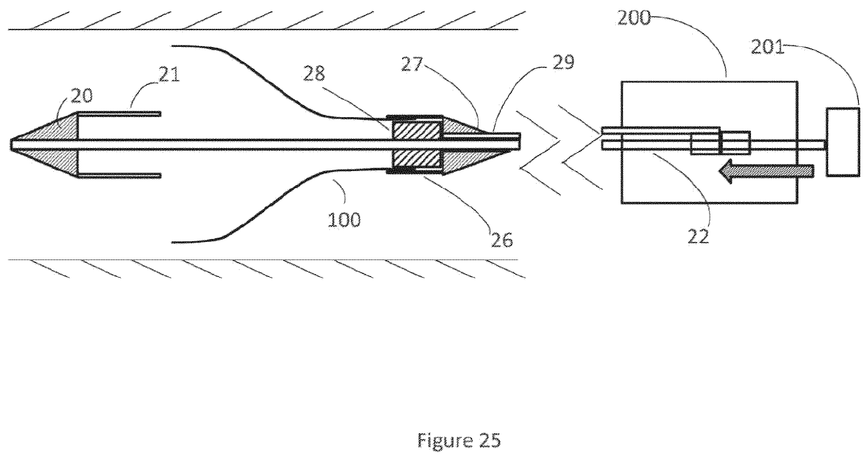

In FIG. 25, the arrow indicates the movement during deployment and the release procedure. Accordingly, a part connected to the first sleeve (22) is pushed distally and actuates the release of the prosthesis from the distal part of the catheter by way of pushing the first sleeve (21) distally and liberating the distal part of the prosthesis.

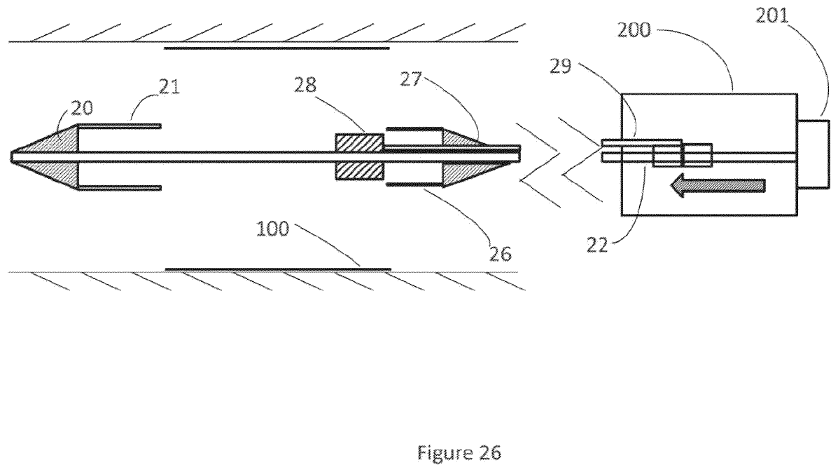

In FIG. 26, the plunger (201) is pushed further distally and thus pushes the stop and shaft (29/29b) distally thus pushing the stent holder (28) distally which leads to the liberation of the proximal part of the prosthesis and its release from the catheter and full release and deployment.

Accordingly FIGS. 21-23 and 24-26 represent the same principle wherein one actuator can actuate two parts of a capsule holding a stent or prosthesis by way of a distal movement of the actuator and a connecting means (29/29a and 29b). In another aspect, the disclosure relates to a specific safety feature in a catheter to lock the loaded prosthesis and to prevent unintentional release from the catheter.

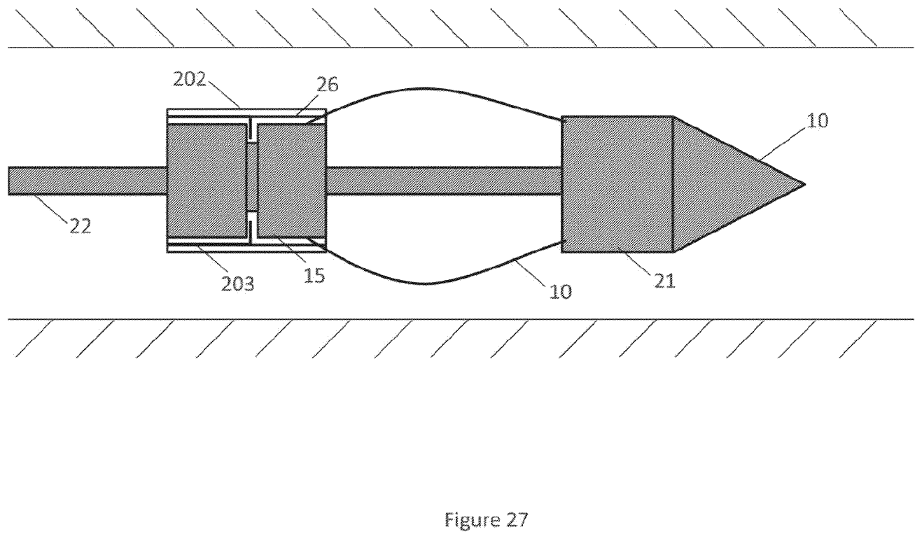

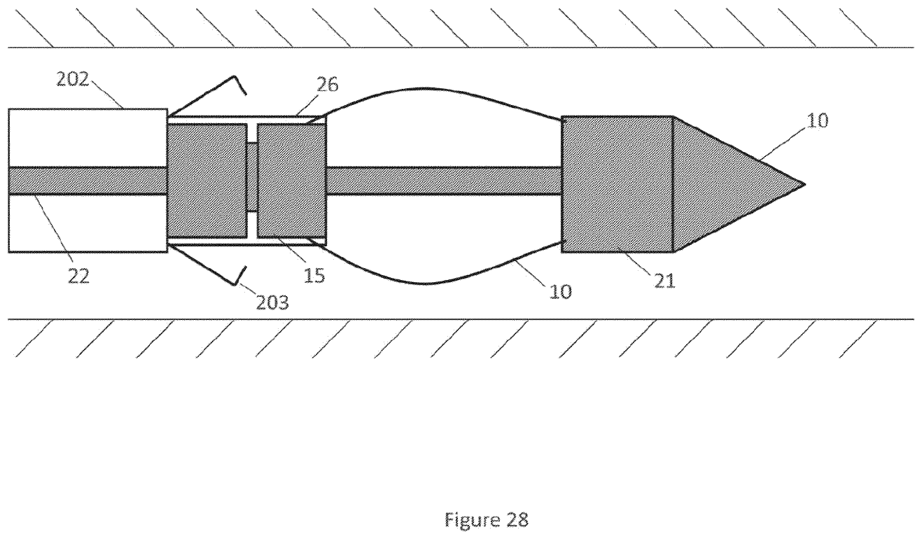

FIG. 27 depicts a safety means designed to avoid premature release of the prosthesis from the catheter by way of a locking clip (203). Prosthesis (103) is held inter alla by second sleeve (26) on stent holder (15) which is characterized by a slit designed to be accessible by locking clip (203). First sleeve (21) holds the distal part of the prosthesis (103). When a locking sleeve (202) is pushed distally it effects opening of the locking clip (203) and radial movement of same (cf. FIG. 28).

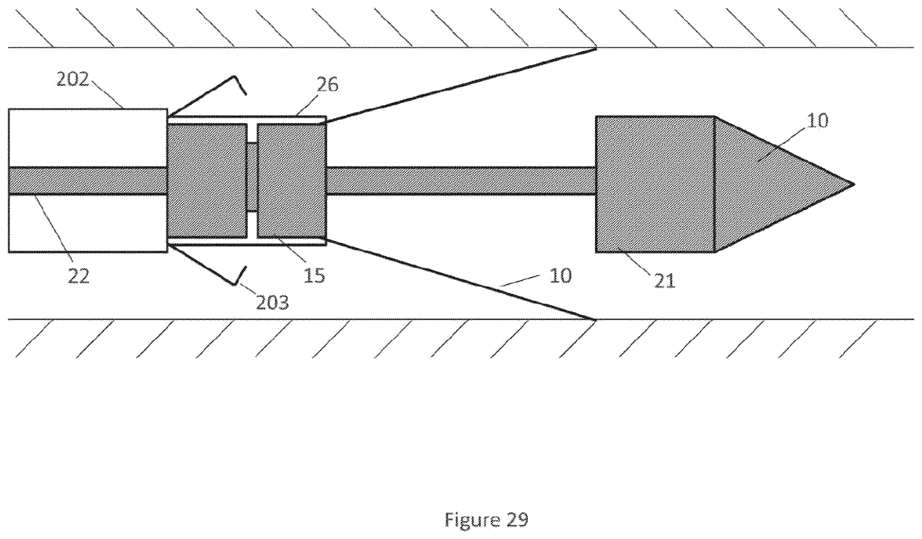

FIG. 29 shows that only after the opening of the locking clip (203) the distal tip and the first sleeve (21) can be pushed distally by way of distal movement of first shaft (22) and the release of the distal part of the prosthesis is effected by way of distal movement of first shaft (22) and first sleeve (21).

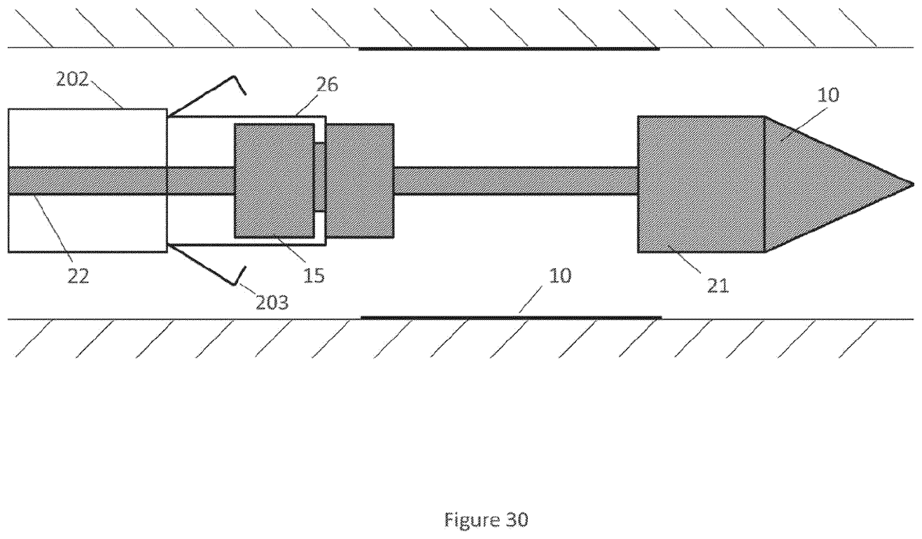

FIG. 30 shows further distal movement of first shaft (22) whereby the stent holder (15) is moved distally and the second sleeve (26) does no longer cover the proximal part of the prosthesis leading to its release and complete release from the catheter tip capsule.

In another aspect the disclosure relates to a loading device particularly useful for loading a transcatheter heart valve prosthesis on a catheter.

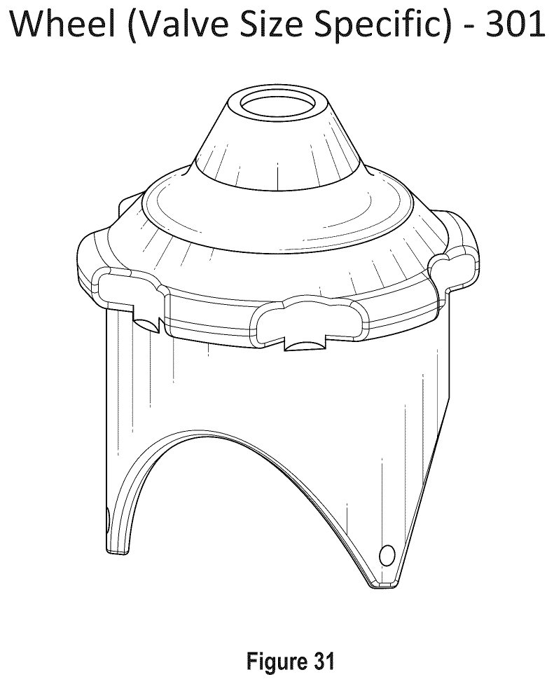

FIGS. 31-36 depict a loading tool for loading a prosthesis onto a catheter capsule for delivery to a patient or into a transport container, The loading part of FIG. 31 is meant to collapse the eyelets so that the eyelets can be captured using axial force generated from rotational force; it also collapses the sealing ring so that the sealing ring can be captured using axial force generated from rotational force.

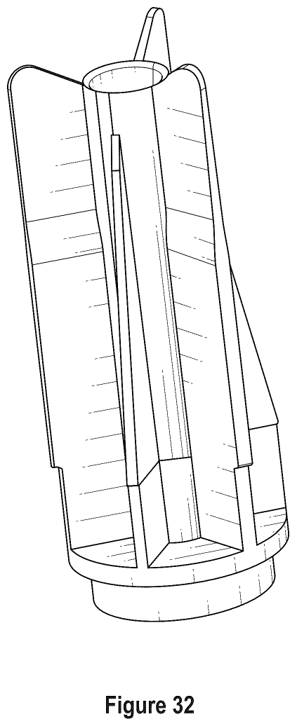

In FIG. 32, a loading part is depicted wherein it provides a base to support the valve during eyelet crimping and during sealing ring extrusion.

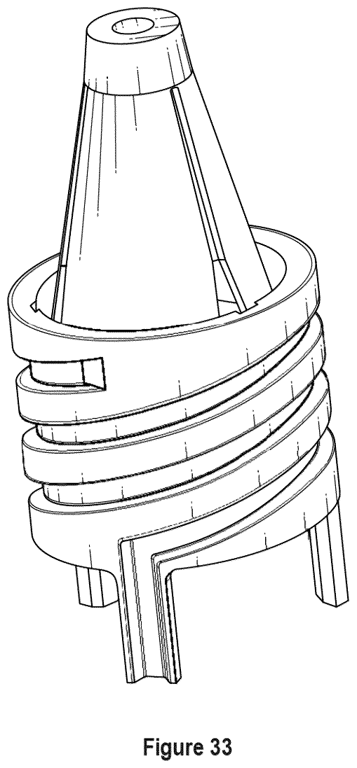

FIG. 33 shows a loading part which provides a track for wheel during eyelet crimping and sealing ring extrusion; moreover it provides contour to collapse sealing ring.

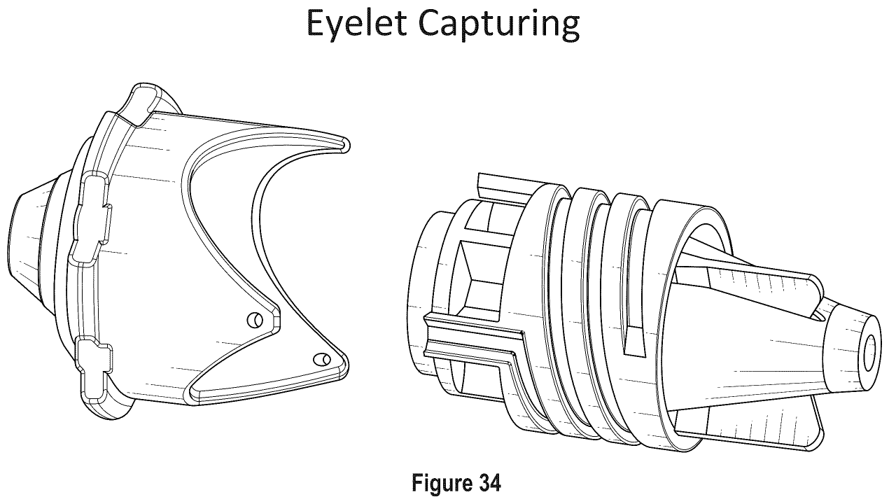

FIG. 34 depicts two parts which are combined together, The prosthesis is placed onto the bottom of one part and the second part collapses the top of the stent of the prosthesis via the interaction between the two parts; this prepares the prosthesis to be mounted onto catheter.

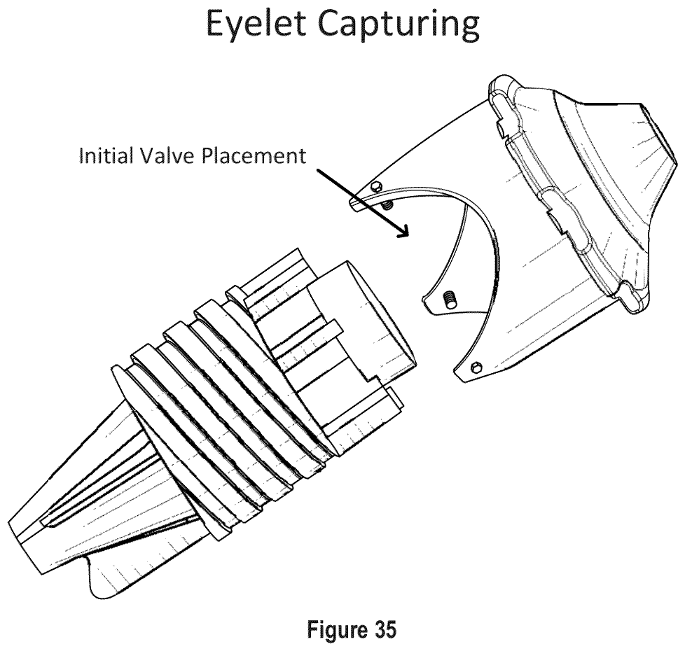

FIG. 35 depicts the parts by which the eyelets of prosthesis are radially crimped as the wheel is pushed down and rotated along the cone.

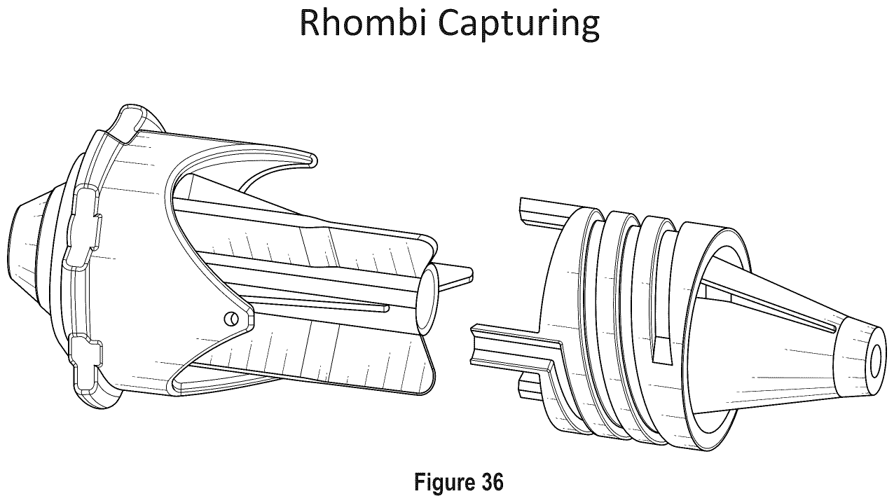

FIG. 36 shows the combination of the two parts which are combined and mounted onto the catheter with the prosthesis sticking outside the top. The assembly is translated to a further part via the threads and crimps the rhombi.

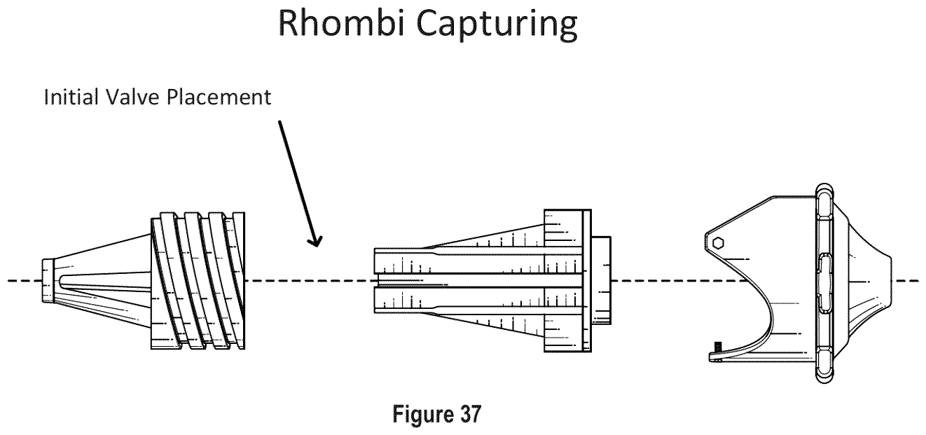

FIG. 37 depicts a step wherein the sealing ring of the prosthesis is radially crimped as the wheel is pushed against the core and rotated along the cone.

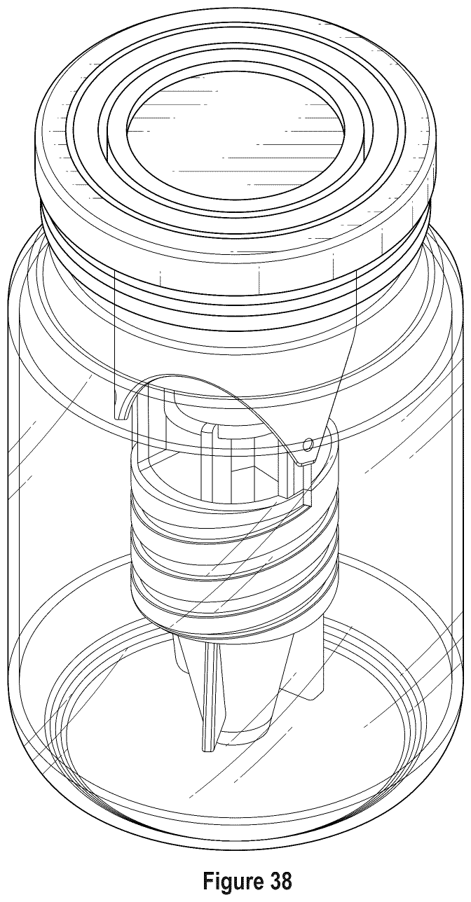

In FIG. 38, the loader and prosthesis are stored together, preferably in a suitable liquid.

### REFERENCE NUMBERS

10 nose cone
11 first sleeve
12 first shaft/first catheter shaft
13 front stop
14 second catheter shaft
15 stent holder
16 second sleeve
17 back cone
20 nose cone
21 first sleeve
22 first shaft
23 front stop
24 passage in front stop
25 catheter shaft (second shaft)
26 second sleeve
27 back mount
28 stent holder
29/29a cable (pull wire)
29/29b shaft for pushing
30 target vessel??? Nose cone???
31 first sleeve
32 first shaft
33 front stop
35 second catheter shaft
36 second sleeve
37 back mount
38 stent holder
39 spring
100 stent
101 second end of stent
102 first end of stent
103 bioprosthetic valve (preferably pericard valve)
104a-c eyelets (preferably to secure the stent in the catheter)
105 skirt
105a-c eyelets of the stent
200 handle
201 plunger
202 locking sleeve
203 locking clips
300 valve prosthesis
302 proximal end
303 midsection
304 tissue leaflets
310 nose cone
311 first sleeve
312 first shaft
313 front stop
315 stent holder
316 second sleeve
317 back cone
318 second shaft
400 container
401 distal holder
402 proximal holder
403 lid
450 container
451 distal holder
452 rod
460 delivery system
461 guidewire lumen
510 distal segment
511 outer shaft
520 delivery system
521 outer shaft
S22 inner shaft
531 outer connector
532 inner connector
601 stent holder
602 wire or string
603 nose cone
604 Inner shaft
605 first sleeve
606 second sleeve
607 outer shaft
610 proximal end
611 outer shaft
612 inner shaft
700 delivery system
701 stent holder
702 wire or string
703 nose cone
705 first sleeve
707 outer shaft
710 proximal end
711 outer shaft
712 inner shaft
801 wheel
802 core
803 cone

What is claimed is:

1. A loading assembly for crimping a prosthetic heart valve and loading the prosthetic heart valve into a distal end of a catheter, the loading assembly comprising:
a first loading part comprising a track and a cone; and
a second loading part comprising a wheel with a plurality of protrusions and a conical portion forming a void at an end of the first loading part, the wheel with the plurality of protrusions configured to interface with the track such that the wheel rotates along the track, (29/29awherein the first loading part and the second loading part are configured to receive the prosthetic heart valve and the second loading part is configured to cause eyelets disposed at a first end of the prosthetic heart valve to collapse as the wheel rotates with respect to the track of the first loading part such that the eyelets are configured to be captured by the distal end of the catheter.

2. The loading assembly of claim 1, further comprising a third loading part configured to receive a portion of the prosthetic heart valve and further configured to be positioned between the wheel and the cone.

3. The loading assembly of claim 2, wherein the third loading part is configured to move axially with respect to the first loading part as the second loading part rotates along the track to cause a sealing ring disposed at a second end of the prosthetic heart valve to collapse such that the sealing ring is configured to be captured by the distal end of the catheter.

4. The loading assembly of claim 3, wherein the cone of the first loading part is contoured to cause the sealing ring to collapse as the second loading part rotates along the track.

5. The loading assembly of claim 2, wherein the cone comprises a plurality of slits.

6. The loading assembly of claim 5, wherein the third loading part comprises a plurality of fins and the plurality of fins are configured to extend through the plurality of slits when the third loading part is positioned between the wheel and the cone.

7. The loading assembly of claim 2, wherein the third loading part is configured to move axially as the second loading part rotates along the track.

8. The loading assembly of claim 2, wherein at least the first loading part and the second loading part together are configured to collapse the first end of the prosthetic heart valve and the first loading part, second loading part, and third loading part together are configured to collapse a second end of the prosthetic heart valve.

9. The loading assembly of claim 1, wherein rotation of the wheel results in rotational force that is applied to eyelets as axial force to cause the eyelets to collapse as the wheel rotates.

10. The loading assembly of claim 1, wherein the second loading part comprises three protrusions that extend in a direction orthogonal to a primary axis of the second loading part.

11. A method for crimping a prosthetic heart valve using a loading assembly and loading the prosthetic heart valve on a distal end of a catheter, the method comprising:
positioning a first end of a prosthetic heart valve into a first loading part of the loading assembly comprising a track and a cone, the first end of the prosthetic heart valve comprising a sealing ring;
positioning a second loading part of the loading assembly onto the first loading part such that a portion of the second loading part is configured to interface with the track of the first loading part, the second loading part comprising a wheel and a conical portion forming a void at an end of the second loading part;
rotating the wheel of the second loading part such that the second loading part moves along the track of the first loading part and causes at least a portion of the prosthetic heart valve to collapse; and
mounting, after causing the at least a portion of the prosthetic heart valve to collapse, the at least a portion of the prosthetic heart valve on the distal end of the catheter.

12. The method of claim 11, wherein a second end of the prosthetic heart valve comprises eyelets and the at least a portion of the prosthetic heart valve that is caused to collapse is the second end of the prosthetic heart valve.

13. The method of claim 11, further comprising positioning a third loading part around at least a second end of the prosthetic heart valve.

14. The method of claim 13, wherein at least a portion of the third loading part is positioned between the first loading part and second loading part when the second loading part is positioned onto the first loading part.

15. The method of claim 14, wherein the cone of the first loading part comprises a plurality of slits.

16. The method of claim 15, wherein the third loading part comprises a plurality of fins and the plurality of fins are configured to extend through the plurality of slits of the cone when the third loading part is positioned between the first loading part and the second loading part.

17. The method of claim 15, wherein a second portion of the prosthetic heart valve is configured to stick outside of the slits of the cone of the first loading part.

18. The method of claim 13, wherein the at least a portion of the prosthetic heart valve that is caused to collapse is the first end.

19. The method of claim 18, wherein rotating the wheel of the second loading part when the second loading part is positioned onto the first loading part causes the third loading part to move axially with respect to the first loading part.

20. The method of claim 11, wherein the track forms multiple rotations around the first loading.
